(19) United States
(12) Patent Application Publication
(10) Pub. No.: US 2011/0238131 A1
(43) Pub. Date: Sep. 29, 2011

(54) LEG ELECTRIC SHOCK SWITCH OF ELECTRONIC WALKING AID

(76) Inventors: Feng-Ching TU, Taipei City (TW); Pin-Hsuan Hung, Taipei City (TW)

(21) Appl. No.: 12/729,600

(22) Filed: Mar. 23, 2010

(51) Int. Cl. A61N 1/00

(52) U.S. Cl. 607/49

(57) ABSTRACT

A leg electric shock switch of an electronic walking aid comprises an upper insulating plate having a restoring resilience; an upper electrode plate, installed below the upper insulating plate; a lower insulating plate; a lower electrode plate, installed above the lower insulating plate; an isolating element, being a plate made of an insulating material, and coupled between the upper electrode plate and the lower electrode plate, and including a plurality of through holes; such that if the upper insulating plate is touched and pressed, the upper electrode plate will produce a deformation, and the upper electrode plate will be passed through the through hole and electrically contacted and connected to the lower electrode plate. Therefore, the manufacturing procedure is simplified and the cost is lowered to achieve the effects of improving the cost-effectiveness of the manufacture, promoting the application of low-cost consuming materials, and enhancing the product competiveness.

### BACKGROUND OF THE INVENTION

1. Field of the Invention

The present invention relates to an electronic walking aid, and more particularly to a leg electric shock switch of an electronic walking aid that provides a cost-effective application that can be promoted extensively.

2. Description of the Related Art

Electronic walking aid is generally used for assisting, rehabilitating or treating a foot drop patient. The main cause of a foot drop is a problem (such as a strike) occurred at the patient's brain or nerves, such that neural signals cannot be transmitted to positions of related muscles of the patient's leg, and thus the patient's foot cannot be lifted normally and the patient has mobility difficulties. Wearing the electronic walking aid can assist a foot-drop patient to walk stably and achieve the effects of reducing the risk of being stumbled while walking, releasing the level of convulsion of thigh and upper leg muscles (quadriceps), increasing the movement range of ankles, and improving a patient's postures, or even providing a function of curing the patient completely. With reference to FIGS. 1, 2 and 2A, an electronic walking aid comprises a main system 80, an electrode pad 81 and a switch 90, wherein the main system 80 is worn at a waist belt of a patient 100 (or worn at any other appropriate position or placed in a carrying bag), and the electrode pad 81 is connected to the main system 80 by a conductive wire 82 and attached to a specific position of a foot-drop leg 101 (such as a shank) of the patient 100, and the switch 90 is connected to the main system 80 by a conductive wire 83 and installed in a shoe 103 worn by a normal leg 102 of the patient 100, such that the switch 90 is situated at the normal leg 102 and below the ankle of the patient 100. The switch 90 comprises an upper insulating plate 91, a lower insulating plate 92 and a plurality of press switches 93 installed between the upper insulating plate 91 and the lower insulating plate 92, wherein the press switch 93 is comprised of a curved upper conducting plate 94 and a lower conducting plate 95, and an area around the upper conducting plate 94 is contacted and electrically insulated with the lower conducting plate 95, and the lower conducting plate 95 is electrically coupled to the conductive wire 96.

During use, if the normal leg 102 of the patient 100 touches and presses the upper insulating plate 91, and the curved portion of the upper conducting plate 94 is pressed and deformed to touch the lower conducting plate 95 to form an electric connection, and a power source is electrically conducted, such that the main system 80 generates a weak current to the electrode pad 81, and a shank muscle of the foot-drop leg 101 is contracted to lift up the leg 101.

Although the foregoing conventional electronic walking aid can achieve the effect of stimulating muscles by electric currents to produce limb movements, the structural design still has the following drawbacks. Since the plurality of press switches 93 are very small, and their operation is achieved by pressing and deforming the curved portion of the upper conducting plate 94 to touch the lower conducting plate 95, therefore the pressure-resisting and elastically deforming capabilities of the upper conducting plate 94 become the key points of the design of the press switches 93, and the press switches 93 must be made of a noble metal with a high strength for resisting pressure and deformation. As a result, the press switches 93 incur a high cost and thus cannot be used extensively as a low-cost consuming material. Obviously, the design of the conventional press switches 92 is not good enough. Since the press switches 93 have a small size, the installation of the press switches 93 and the serial connection of the conductive wire 96 will be very complicated, and thus incurring a higher manufacturing cost. Therefore, it is an important subject for related manufacturers to improve the structure of the conventional electronic walking aid.

In view of the shortcomings of the prior art, the inventor of the present invention based on years of experience in the related industry to conduct extensive researches and experiments, and finally developed an easy-to-install, stable and cost-effective electronic walking aid switch in accordance with the present invention, in hope of serving the general public and promoting the development of the related industry.

### SUMMARY OF THE INVENTION

Therefore, it is a primary objective of the present invention to provide a leg electric shock switch of an electronic walking aid that provides a simple and easy way of manufacturing a stable and secured product and achieves the effects of lowering the manufacturing cost, promoting an extensive application, and enhancing the competitiveness of the product.

To achieve the foregoing objective, the present invention discloses a leg electric shock switch of an electronic walking aid comprising:

an upper insulating plate, having a restoring resilience;

an upper electrode plate, installed below the upper insulating plate;

a lower insulating plate;

a lower electrode plate, installed above the lower insulating plate;

an isolating element, being a plate made of an insulating material, and coupled between the upper electrode plate and the lower electrode plate, and including a plurality of through holes;

thereby, if the upper insulating plate is touched and pressed, the upper electrode plate will produce a deformation, and the upper electrode plate will be passed through the through hole and electrically contacted and connected to the lower electrode plate.

The technical characteristics and effects of the present invention will become apparent with the detailed description of the preferred embodiments and the illustration of the related drawings.

### BRIEF DESCRIPTION OF THE DRAWINGS

FIG. 1 is a schematic view of an application of a conventional electronic walking aid;

FIG. 2 is a cross-sectional view of a switch of a conventional electronic walking aid;

FIG. 3 is an enlarged view of a portion of a switch of a conventional electronic walking aid;

FIG. 3 is an exploded view of the present invention;

FIG. 4 is a perspective view of the present invention;

FIG. 4A is a cross-sectional view of the present invention;

FIG. 5A is a schematic view of a first application of the present invention; and

FIG. 5B is a schematic view of a second application of the present invention.

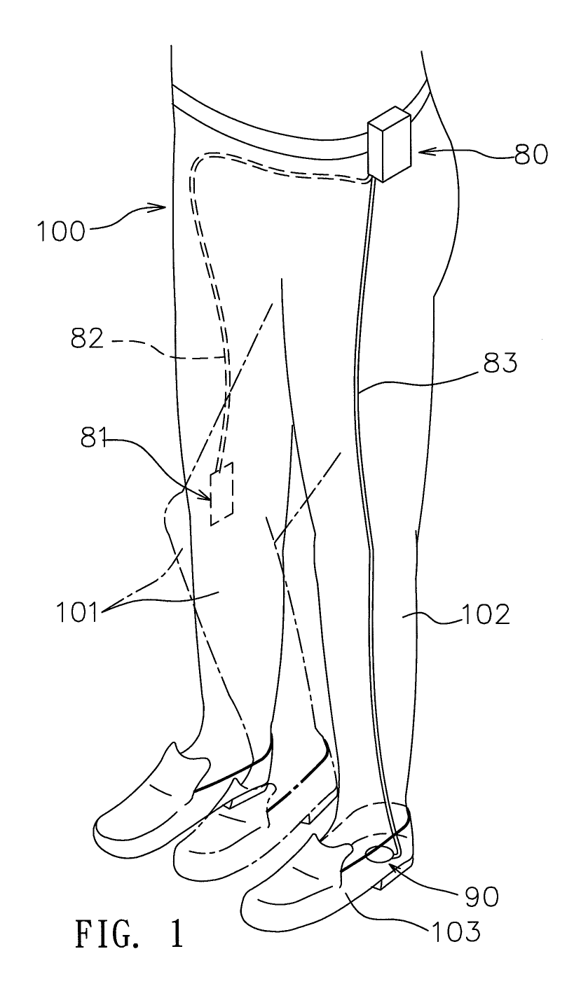

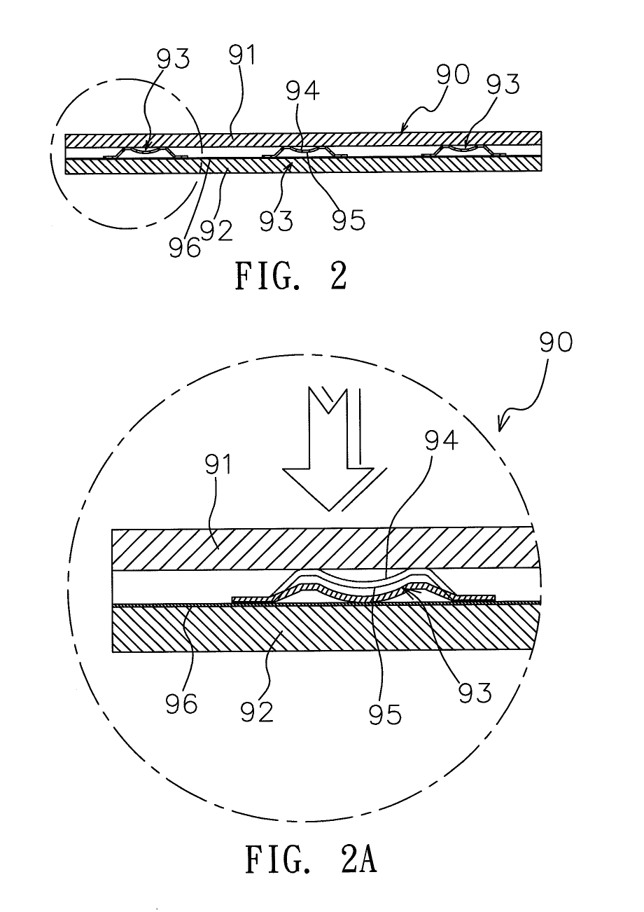

### DETAILED DESCRIPTION OF THE PREFERRED EMBODIMENTS

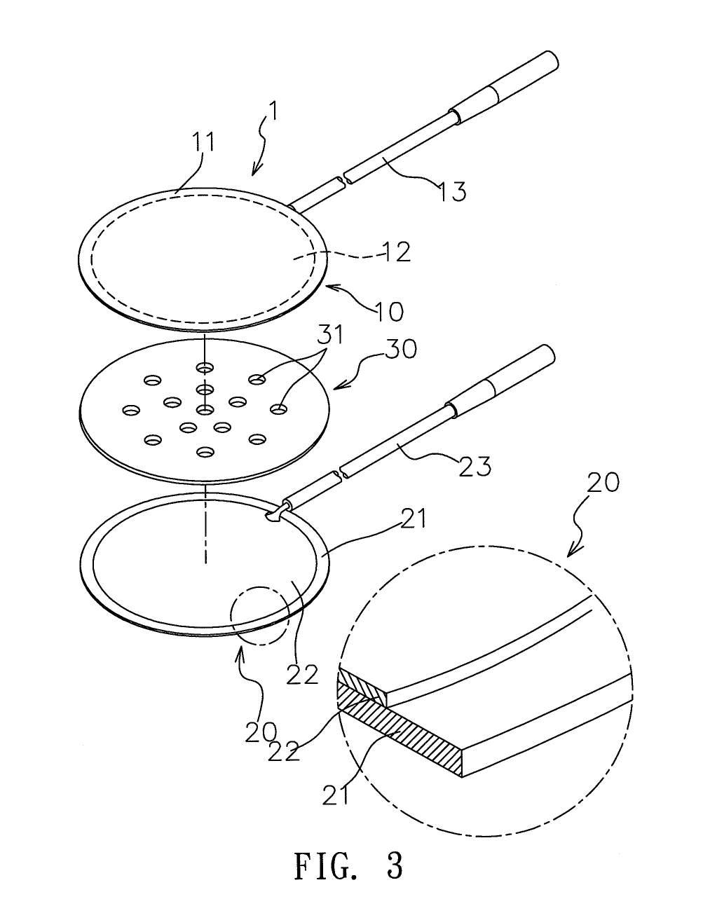

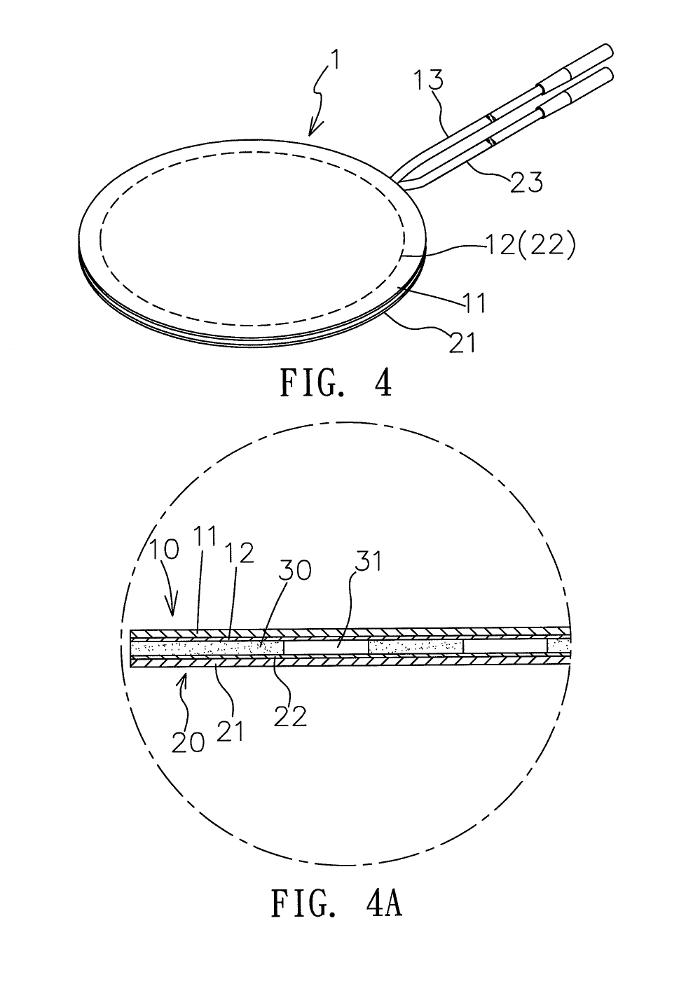

With reference to FIGS. 3, 4 and 4A for a leg electric shock switch 1 of an electronic walking aid switch 1 of the present invention, the switch 1 comprises an upper component module 10, a lower component module 20 and an isolating element 30. The upper component module 10 comprises an upper insulating plate 11 and an upper electrode plate 12, wherein the upper insulating plate 11 has a restoring resilience, and the upper electrode plate 12 is installed below the upper insulating plate 11, and the upper electrode plate 12 generally has an area smaller than the area of the upper insulating plate 11, such that the upper insulating plate 11 can be covered completely and protruded from an external side of the upper electrode plate 12, and the upper electrode plate 12 is connected to a main system (not shown in the figure) by an electrode wire 13. The lower component module 20 comprises a lower insulating plate 21 and a lower electrode plate 22, wherein the lower electrode plate 22 is installed above the lower insulating plate 21, and the lower electrode plate 22 generally has an area smaller than the area of the lower insulating plate 21, such that the lower insulating plate 21 can be received completely and protruded out from an external side of the lower electrode plate 22, and the lower electrode plate 22 is connected to the main system by an electrode wire 23, and the electrode wires 13, 23 form an electrode circuit. The isolating element 30 is a plate made of an insulating material and having appropriate thickness, flexibility and elasticity, and the isolating element 30 is installed between the upper component module 10 (or the upper electrode plate 12) and the lower component module 20 (or the lower electrode plate 22), and the isolating element 30 includes a plurality of through holes 31. In an embodiment, the isolating element 30 has an area equal to or greater than the area of the upper electrode plate 12 or the area of the lower electrode plate 22, such that the upper electrode plate 12 is isolated from the lower electrode plate 22 completely.

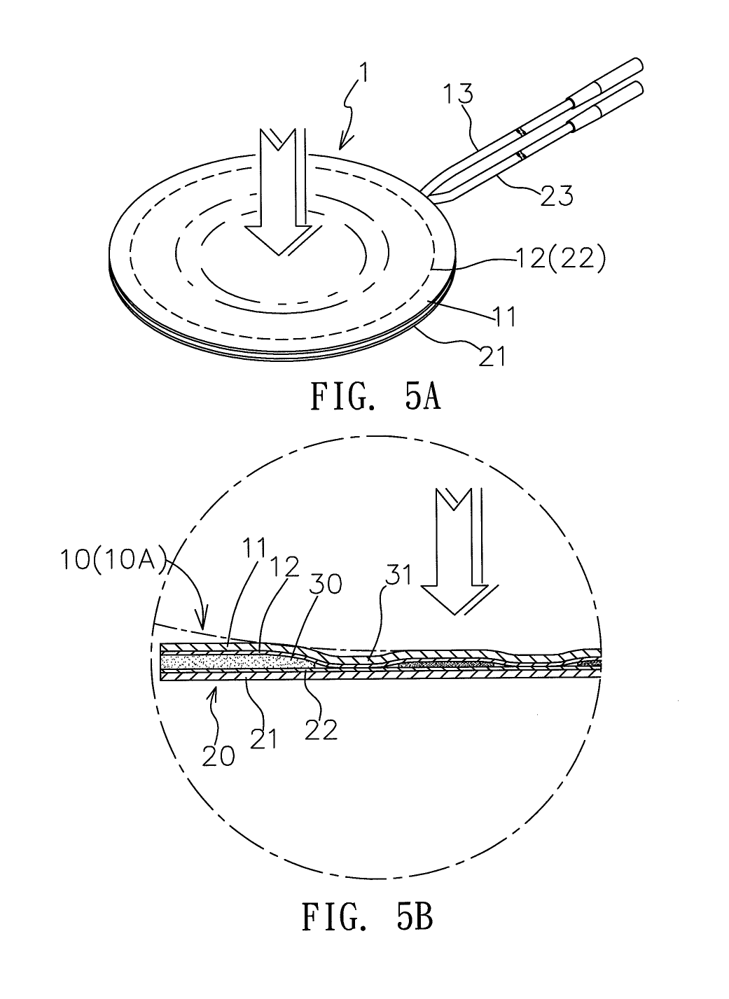

With reference to FIGS. 5A and 5B, if the electronic walking aid switch 1 is not started or the upper component module 10 is not touched and pressed by a patient's foot, the upper electrode plate 12 and the lower electrode plate 22 will be isolated by the isolating element 30 and cannot be contacted with each other or electrically conducted; and if the upper component module 10 is touched and pressed by the patient's foot, then the upper component module 10 will be pressed to produce a deformation 10A of the upper component module 10 corresponding to the through hole 31. In other words, both upper and lower insulating plates 11, 12 are pressed and deformed simultaneously at the deformed position of the upper component module, such that the upper electrode plate 12 is passed through the through hole 31 and contacted with the lower electrode plate 22 to electrically connect a power source, and the main system generates a weak current to produce a muscle activity effect of the patient. If the patient's foot is lifted (such that no gravitational force will be provided for the pressing and contact), then the upper component module 10 (including the upper insulating plate 11 and the upper electrode plate 12) will elastically resume its original shape, and the upper electrode plate 12 and the lower electrode plate 22 will resume their original state of being isolated by the isolating element 30.

In summation of the description above, the leg electric shock switch of an electronic walking aid of the present invention adopts the large upper and lower electrode plates 12, 22 for the pressing, contact, and electric connection, so that the invention does not require the strict condition of the conventional small switch having a powerful strength to resist pressure, and a general metal material can be used to achieve the desired effect. The present invention not just provides a durable product only, but also improves the cost-effectiveness. In addition, the present invention has a simple, easy and practical design, not just providing high reliability to prolong the lifespan of the product, but also providing an easy manufacturing procedure to lower the cost of the product. In addition, the product can be made of a low-cost material and used extensively, such that the product competitiveness can be enhanced significantly.

The present invention improves over the prior art and complies with patent application requirements, and thus is duly filed for the patent application. While the invention has been described by device of specific embodiments, numerous modifications and variations could be made thereto by those generally skilled in the art without departing from the scope and spirit of the invention set forth in the claims.

What is claimed is:

1. A leg electric shock switch of an electronic walking aid, comprising:
an upper insulating plate, having a restoring resilience;
an upper electrode plate, installed below the upper insulating plate;
a lower insulating plate;
a lower electrode plate, installed above the lower insulating plate;
an isolating element, being a plate made of an insulating material, and coupled between the upper electrode plate and the lower electrode plate, and including a plurality of through holes;
thereby, if the upper insulating plate is touched and pressed, the upper electrode plate will produce a deformation, and the upper electrode plate will be passed through the through hole and electrically contacted and connected to the lower electrode plate.

2. The leg electric shock switch of an electronic walking aid as recited in claim 1, wherein the upper electrode plate has an area smaller than the area of the upper insulating plate.

3. The leg electric shock switch of an electronic walking aid as recited in claim 1, wherein the upper electrode plate is connected to a main system by an electrode wire.

4. The leg electric shock switch of an electronic walking aid as recited in claim 1, wherein the lower electrode plate has an area smaller than the area of the lower insulating plate.

5. The leg electric shock switch of an electronic walking aid as recited in claim 1, wherein the lower electrode plate is connected to the main system by an electrode wire.

6. The leg electric shock switch of an electronic walking aid as recited in claim 1, wherein the isolating element is flexible and elastic.

7. The leg electric shock switch of an electronic walking aid as recited in claim 1, wherein the isolating element has an area equal to or greater than the area of the upper electrode plate or the area of the lower electrode plate.
